(19) United States
(12) Patent Application Publication
(10) Pub. No.: US 2026/0263484 A1
(43) Pub. Date: Sep. 10, 2026

(54) METHODS FOR TREATING CANCERS

(71) Applicant: Yung Kien Industrial Corp., TAIPEI (TW)

(72) Inventors: Kuang-Dee CHEN, New Taipei City (TW); Teng-Hai CHEN, New Taipei City (TW); Cheng-Po HUANG, New Taipei City (TW); Su-Yu CHEN, New Taipei City (TW); Ssu-Chia WANG, New Taipei City (TW); Yi-Hsiu LIN, New Taipei City (TW)

(21) Appl. No.: 19/073,728

(22) Filed: Mar. 7, 2025

(51) Int. Cl. A61K 31/575; A61K 31/337; A61K 39/395; A61P 35/00

(52) U.S. Cl. CPC A61K 31/575; A61K 31/337; A61K 39/3955; A61P 35/00

(57) ABSTRACT

Provided herein is a method for the treatment of a cancer. The method includes administering to a subject a combination of ganoderic acid T (GAT) and an anti-cancer agent to synergistically suppress the growth of the cancer as compared to that achieved by the anti-cancer agent alone. According to embodiments of the present disclosure, GAT may be administered prior to, together with, or after the administration of the anti-cancer agent.

### STATEMENT REGARDING PRIOR DISCLOSURES BY THE INVENTOR OR A JOINT INVENTOR UNDER 37 C.F.R. 1.77(B)(6)

Part of the subject matter of the invention described in the present application was published by the inventors, Kuangdee Chen, Denghai Chen, Chengpo Huang, Suyu Chen, Ssuchia Wang and Yihsiu Lin in an article titled “Ganoderic acid T, a i Ganoderma triterpenoid, modulates the tumor microenvironment and enhances the chemotherapy and immunotherapy efficacy through downregulating galectin-1 levels.” The article was published in Toxicology and Applied Pharmacology 2024, 491:117069. The publication was made by and/or originated from 6 members of the inventive entity of the present invention, and the entirety of this article is incorporated herein by reference. A copy of the article is provided in a concurrently filed Information Disclosure Statement pursuant to the guidance of 78 Fed. Reg. 11076 (Feb. 14, 2013).”

### BACKGROUND OF THE INVENTION

### 1. Field of the Invention

The present disclosure in general relates to cancer treatment, particularly, the present disclosure relates to a method of enhancing the susceptibility of a cancer toward an anti-cancer agent by administering ganoderic acid T (GAT) prior to, together with or after administering the anti-cancer agent to the subject.

### 2. Description of Related Art

Ganoderma lucidum, commonly known as Lingzhi or Reishi, is a medicinal mushroom used in traditional medicine, particularly in Asian countries. It is widely used for its potential health benefits, including anti-cancer properties. G. lucidum has gained attention as a potential cancer therapeutic agent owing to its rich bioactive constituents, such as polysaccharides, proteins, and triterpenoids, with various biological activities, such as immunomodulatory, antioxidant, and anti-tumor activities.

Studies on the cytotoxic effects of G. lucidum on cancer cells have mainly focused on ganoderic triterpenes. Among these triterpenes, ganoderic acid T (GAT) has been identified as one of the active compounds. GAT is present in the mycelium at a concentration of 4.86% based on the mycelial dry weight. Due to its high concentration, GAT can be purified as a single entity and holds the potential for development into a cancer therapeutic.

Inventors of the present disclosure unexpectedly discovered GAT could enhance the susceptibility of cancer toward anti-cancer agents and thereby increasing intratumoral drug concentrations and reducing tumor size, thus GAT may serve as candidates for the development of a medicament for improving the therapeutic effect of chemotherapy or immunotherapy for cancers.

### SUMMARY

Accordingly, it is the aspect of this disclosure to provide a method of treating a cancer in subject. The method includes administering to the subject an effective amount of a combination of ganoderic acid T (GAT) and an anti-cancer agent, wherein the administration of the combination of GAT and the anti-cancer agent results in a synergistic suppression of the growth of the cancer as compared to that achieved by the anti-cancer agent alone.

According to embodiments of the present disclosure, the GAT is administered before, together with, or after administering the anti-cancer agent, so as to synergistically suppress the growth of the cancer.

According to embodiments of the present disclosure, the anti-cancer agent is a chemotherapeutic agent or an anti-PD-L1 antibody.

Examples of the chemotherapeutic agent suitable for use in the present method include, but are not limited to, paclitaxel, docetaxel, camptothecin (CPT), topotecan (TPT), irinotecan (CPT-11), doxorubicin, daunorubicin, epirubicin, fluorouracil, cisplatin, cyclophosphamide, vinblastine, vincristine, ifosfamide, melphalan, mitomycin, methotrexate, mitoxantrone, teniposide, etoposide, bleomycin, leucovorin, cytarabine, dactinomycin, streptozocin, and combretastatin A4-phosphate. Preferably, the chemotherapeutic agent is paclitaxel.

Example of the anti-PD-L1 antibody suitable for use in the present method includes, but is not limited to, atezolizumab.

Examples of the cancer suitable to be treated by the present method include, but are not limited to, glioblastoma, thyroid cancer, gastrointestinal cancer, liver cancer, lung cancer, breast cancer, pancreatic cancer, melanoma, prostate cancer, ovarian cancer, adenocarcinoma, bladder cancer and kidney cancer. In some embodiments of the present disclosure, the cancer is ovarian cancer. In other embodiments of the present disclosure, the cancer is breast cancer.

According to some embodiments of the present disclosure, the GAT and paclitaxel are respectively administered at 8 mg/Kg and 2 mg/Kg to the subject.

According to other embodiments of the present disclosure, the GAT and atezolizumab are respectively administered at 8 mg/Kg and 0.8 mg/Kg to the subject.

In all embodiments of the present disclosure, the subject is a human.

The details of one or more embodiments of this disclosure are set forth in the accompanying description below. Other features and advantages of the invention will be apparent from the detail descriptions, and from claims.

### BRIEF DESCRIPTION OF THE DRAWINGS

The present description will be better understood from the following detailed description read in light of the accompanying drawings, where:

FIG. 1. The iidentification of ganoderic acid T (GAT). High-performance liquid chromatography (HPLC) revealed the (A) eluted peaks of crude GAs for the identification of individual GAs as well as the (B) peak of GAT;

FIG. 2 The cytotoxicity of GAT in cancer cell lines. Dose-escalation effects of GAT on cell survival rate in various cancerous cell lines. ES-2 (▪), EMT6 (♦), AGS(▴), and AsPC1 (•) cells were treated with GAT at the indicated concentrations for 72 hours and cell survival rate was assessed by 3-(4,5-dimethylthiazol-2-yl)-2,5-diphenyltetrazolium bromide (MTT) assay. Each in vitro experiment was carried out at least three times in triplicate. Points, mean (n=3); bars, SD.

FIG. 3 Tumor responses and infiltration of immune cells in orthotopic ovarian tumors after oral administration of GAT to humanized peripheral blood mononuclear cell (Hu-PBMC)-mice. Each humanized or non-humanized mouse was injected 1×10⁵ ES-2 cells into the left ovarian bursa and treated with the vehicle or 100 mg/kg GAT via oral gavage daily during the study period. (A) Tumor weights, (B) α-SMA-positive area, and (C) galectin-1 (Gal-1) intensity was quantified in orthotopic ovarian tumors. Columns, mean (n=3); bars, SD. *P<0.05. (D) Percentages of tumor-infiltrating lymphocytes (TILs) in vehicle or GAT-treated humanized mice were determined via flow cytometry. Columns, mean (n=3); bars, SD. *P<0.05.

FIG. 4. Gal-1 mediates GAT-induced cell cytotoxicity via phospho-extracellular signal-regulated kinase (p-ERK) regulation. (A) GAT induced Gal-1 and p-ERK inactivation in a dose-dependent manner. ES-2 cells were treated with GAT at the indicated concentration for 48 hours. The normalized quantitative results are presented as relative intensity below the blot. (B) Dose-dependent effects of GAT on EMT6, AGS, and AsPC1 cells. (C) Gal-1 reverses the cell cytotoxicity of GAT. ES-2 cells overexpressing Gal-1 with His-tag were treated with 50 μg/mL GAT for 72 hours. Overexpression of Gal-1 in ES-2 cells was verified via western blotting (top); the cell survival rate was analyzed by MTT assay (bottom). Columns, mean (n=3); bars, SD. *P<0.05 and *P*<0.01.

FIG. 5 GAT promotes Gal-1 ubiquitination in ES-2 cells. (A) The protein expression of Gal-1 in ES-2 cells. ES-2 cells were treated with GAT at the indicated concentrations or MG132 (10 μM) for 48 hours. High M.W., high molecular weight. The normalized quantitative results are presented as relative intensity below the blot. (B) The degradation of Gal-1 by GAT induces ubiquitination. GAT-treated ES-2 cell lysates were immunoprecipitation (IP) with the Gal-1 antibody and immunoblotting (IB) with the ubiquitin-specific antibody. (C) Selective inhibition of Gal-1 by GAT through in vitro proteasome assay. Whole cell extracts were added with proteasome assay buffer, GAT (0-40 μg/mL), and ATP (2 mM) at 37° C. for 72 hours.

FIG. 6. Effects of GAT on in vitro and in vivo models. (A) The combination index of three paclitaxel combined treatments. ES-2 cells were treated with three paclitaxel combinations with cisplatin (□), doxorubicin (○), or GAT (Δ) for 72 hours, and the combination index plots were generated using the CompuSyn software. TAX: paclitaxel, DOX: doxorubicin, Cis: cisplatin, Fa: Fraction affected, and CI: combination index. (B) The tumor weights of orthotopic ovarian ES-2 tumors in NPG mice were treated with GAT and paclitaxel (left). Each mouse was inoculated with 1×10⁵ ES-2 cells and treated with the vehicle or 100 mg/kg GAT via oral gavage daily and/or paclitaxel 20 mg/kg i.v. once weekly. High levels of paclitaxel were detected in tumors along with GAT by LC-MS/MS system (right). Columns, mean (n=4); bars, SD. *P<0.05 and **P<0.01.

FIG. 7 In vivo effect of GAT on immunotherapy efficacy in the EMT6 syngeneic mammary tumor model. (A) Effects of GAT and/or anti-programmed death-ligand 1 (PD-L1) on tumor growth. Each mouse was inoculated subcutaneously in the dorsal flank with 5×10⁵ EMT6 cells and treated with 20 mg/kg GAT i.v. twice a week and/or 10 mg/kg anti-PD-L1 i.p. once weekly for the duration of the study. Points, mean (n=4); bars, SD. *P<0.05, **P<0.01, and ***P<0.001. (B) Tumor weights were quantified in EMT6 tumors. Columns, mean (n=4); bars, SD. *P<0.05 and ***P<0.001. (C, D, E) Quantitative analysis of α-SMA, Gal-1, and CD 8. Columns, mean (n=4); bars, SD. *P<0.05 and **P<0.01.

### DETAILED DESCRIPTION OF PREFERRED EMBODIMENTS

The detailed description provided below in connection with the appended drawings is intended as a description of the present disclosure and is not intended to represent the only forms in which the present disclosure may be constructed or utilized.

### 1. Definition

For convenience, certain terms employed in the context of the present disclosure are collected here. Unless defined otherwise, all technical and scientific terms used herein have the same meaning as commonly understood by one of the ordinary skill person in the art to which this invention belongs.

The terms “treatment” and “treating” are used herein to include treatment that results in a desired pharmaceutical and/or physiological effect. Preferably, the effect is therapeutic in terms of partially or completely curing or preventing the growth of tumor cells. Also, the term “treating” as used herein refers to application or administration of a compound or an agent of the present disclosure to a subject, who has a medical condition, a symptom of the condition, a disease or disorder secondary to the condition, or a predisposition toward the condition, with the purpose to partially or completely alleviate, ameliorate, relieve, delay onset of, inhibit progression of, reduce severity of, and/or reduce incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. As used herein, the symptom, disease, disorder or condition may be solid tumor or metastatic tumor. Treatment is generally “effective” if one or more symptoms or clinical markers are reduced as that term is defined herein.

The term “an effective amount” as used herein refers to an amount effective, at dosages, and for periods of time necessary, to achieve the therapeutically desired result with respect to the treatment of cancer. According to embodiments of the present disclosure, the effective amount may be human equivalent dose (HED) calculated from animal dose in accordance with the Guidance for Industry issued by the Food and Drug Administration (FDA), Center for Drug Evaluation and Research (CDER) in U.S.A.

The terms “administered,” “administering” and “administration” are used interchangeably herein to refer means either directly administering a compound or a combination of compounds of the present invention, which will produce therapeutic effect within the body.

The term “subject” or “patient” refers to an animal including the human species that is treatable with the compositions and/or methods of the present invention. The term “subject” or “patient” intended to refer to both the male and female gender unless one gender is specifically indicated. Accordingly, the term “subject” or “patient” includes, but is not limited to, human, non-human primate such as any mammal, dog, cat, horse, sheep, pig, cow, and etc., preferably a human, which may benefit from treatment by the combination of compounds of this disclosure. The terms “subject” and “patient” are used interchangeably in the present disclosure.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

### 2. The Treatment Method

The present disclosure is based, at least in part, unexpected discovery that ganoderic acid T (GAT) isolated from the fruit bodies or mycelia of Ganoderma lucidum, may enhance the susceptibility of a cancer toward an anti-cancer agent, thus enhances the therapeutic effect of the anti-cancer agent.

Accordingly, it is the first aspect of the present disclosure to provide a method for the treatment of a cancer. The method includes the step of administering to the subject an effective amount of a combination of GAT and an anti-cancer agent to synergistically suppress the growth and/or metastasis of the cancer.

In general, GAT may be isolated from Ganoderma spp in accordance with any method known in the art, such as from the fruit bodies of Ganoderma lucidum in accordance with the method described in the “Materials and methods” section of the present disclosure. Alternatively, GAT may be isolated from the cultivating waste of Ganoderma lucidum, particularly from the mycelium remained in the cultivating media of Ganoderma lucidum after harvest, in accordance with the method described in Taiwan Patent No. I381844, the disclosure of which is incorporated herein by reference. The isolation in general involves extracting the Ganoderma spp with a solvent, preferably an alcoholic solution, at a temperature above room temperature; followed by column chromatography purification, which may be high performance liquid chromatography (HPLC), reverse phase liquid chromatography and etc.; concentrating and drying the isolated product, until a dried powder is produced.

According to embodiments of the present disclosure, the GAT is administered before, together with, or after administering the anti-cancer agent, to synergistically suppress the growth of the cancer.

According to embodiments of the present disclosure, the anti-cancer agent may be a chemotherapeutic agent or an anti-PD-L1 antibody. Examples of the chemotherapeutic agent suitable for use in the present method include, but are not limited to, paclitaxel, docetaxel, camptothecin (CPT), topotecan (TPT), irinotecan (CPT-11), doxorubicin, daunorubicin, epirubicin, fluorouracil, cisplatin, cyclophosphamide, vinblastine, vincristine, ifosfamide, melphalan, mitomycin, methotrexate, mitoxantrone, teniposide, etoposide, bleomycin, leucovorin, cytarabine, dactinomycin, streptozocin, and combretastatin A4-phosphate. In some embodiments of the present disclosure, GAT is administered in combination with paclitaxel to the subject. Example of the anti-PD-L1 antibody suitable for use in the present method includes, but is not limited to, atezolizumab. In other preferred embodiments of the present disclosure, GAT is administered in combination with atezolizumab to the subject.

Examples of the cancer that may be treated by the present method include, but are not limited to, glioblastoma, thyroid cancer, gastrointestinal cancer, liver cancer, lung cancer, breast cancer, pancreatic cancer, melanoma, prostate cancer, ovarian cancer, adenocarcinoma, bladder cancer and kidney cancer. In some embodiments of the present disclosure, the subject has ovarian cancer. In other embodiments of the present disclosure, the subject has breast cancer.

According to embodiments of the present disclosure, to elicit a therapeutic effect in mice, GAT is administered to the subject in an amount of 1 to 100 mg/Kg per dose, and the ant-cancer agent is administered to the subject in an amount of 0.1 to 100 mg/Kg per dose. In some preferred embodiments of the present disclosure, GAT is orally administered to the subject in the amount of 100 mg/Kg, while paclitaxel is administered to the subject in the amount of 20 mg/Kg via intravenous injection. In other preferred embodiments of the present disclosure, both GAT and anti-PD-L1 antibody are administered to the subject via intravenous injection in the amount of 20 mg/Kg and 10 mg/Kg, respectively.

A skilled artisan may readily determine the human equivalent dose (HED) of GAT or the anti-cancer agent based on the animal dose provided in working examples of the present disclosure. The GAT may be administered to a human subject in an amount of about 0.1 to 100 mg/Kg body weight of the subject, such as 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/Kg; preferably, in the amount of about 0.5 to 50 mg/Kg, such as 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 mg/Kg; more preferably, in the amount of 1 to 20 mg/Kg, such as 1, 2, 3, 4, 5, 6, 7, 8, 9, 10, 11, 12, 13, 14, 15, 16, 17, 18, 19, 20 mg/Kg. According to some preferred embodiments of the present disclosure, GAT may be administered to the human subject at the dose of 8 mg/Kg (per os). According to other preferred embodiments of the present disclosure, GAT may be administered to the human subject at the dose of 0.8 mg/Kg (i.p.). According to embodiments of the present disclosure, the anti-cancer agent may be administered to a human subject in an amount of about 0.1 to 100 mg/Kg body weight of the subject, such as 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mg/Kg; preferably, in the amount of about 0.5 to 50 mg/Kg, such as 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/Kg; more preferably, in the amount of 1 to 20 mg/Kg, such as 1.0, 2.0, 3.0, 4.0, 5.0, 6.0, 7.0, 8.0, 9.0, 10, 11, 12, 13, 14, 15, 16, 17, 18, 19, 20 mg/Kg. According to preferred embodiments of the present disclosure, paclitaxel may be administered to the human subject at the dose of 2 mg/Kg (per os). According to other embodiments of the present disclosure, atezolizumab may be administered to the human subject at the dose of 0.8 mg/Kg (i.p.).

It will be appreciated that the dosage of GAT or anti-cancer agent will vary from patient to patient not only for the particular route of administration, and the ability of the GAT or the anti-cancer agent to elicit a desired response in the patient, but also factors such as disease state or severity of the condition to be alleviated, age, sex, weight of the patient, the state of being of the patient, and the severity of the pathological condition being treated, concurrent medication or special diets then being followed by the patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of GAT, paclitaxel or a combination thereof are outweighed by the therapeutically beneficial effects. Preferably, GAT or the anti-cancer agent of the present disclosure is administered at a dosage and for a time such that the number and/or severity of the symptoms are decreased. The dose can be administered in a single dosage, or alternatively in more than one dosage, such as 2, 3, or 4 dosages a day.

In one preferred embodiment, GAT (e.g., at the dose of 8 mg/Kg) is administered together with paclitaxel (e.g., at the dose of 1.60 mg/Kg) to a subject suffering from ovarian cancer. In another preferred embodiment, GAT (e.g., at the dose of 8 mg/Kg) is administered together with atezolizumab (e.g., at the dose of 0.8 mg/Kg) to a subject suffering from breast cancer.

The present invention will now be described more specifically with reference to the following embodiments, which are provided for the purpose of demonstration rather than limitation.

### EXAMPLES

### Materials and Methods

Preparation of Ganoderic Acid T (GAT) from G. lucidum

Ganoderma mycelium powder was ethanol-extracted, centrifuged, and vacuum-dried to produce a crude powder. This powder was then dissolved, and filtered, and the filtrate was examined with high-performance liquid chromatography (HPLC) to identify individual ganoderic acids (GAs). The eluted GAT peak was collected and concentrated in an evaporator for crystallization and identification. Purified GAT was reconstituted in EtOH for in vitro study. The ethanol concentration was 0.25% at a GAT dosage of 50μg/mL. GAT was mixed with microcrystalline cellulose at a 1:2 (w/w) ratio and dissolved in distilled H₂O for oral gavage. For intravenous injection in animal experiments, 40 mg GAT was dissolved in 1 mL of a stock solution containing Cremophor EL and ethanol (1:1), and the stock was diluted with saline before injection resulting in a final ethanol concentration of 5%.

### Cell Culture

ES-2 human ovarian clear cell adenocarcinoma, AGS gastric adenocarcinoma, and AsPC-1 pancreatic ductal adenocarcinoma cell lines were purchased from the Bioresource Collection and Research Center (Hsinchu City, Taiwan). EMT6 mouse mammary cancer cell line was purchased from the American Type Culture Collection (Virginia, USA). ES-2 cells were cultured in Dulbecco's modified Eagle's medium (DMEM). AsPC1 cells were cultured in the Roswell Park Memorial Institute-1640 medium. AGS cells were cultured in the F-12K Medium (Kaighn's modified Ham's F-12 Medium). These culture media were supplemented with 10% fetal bovine serum (FBS), 100 U/mL penicillin, 100 μg/mL streptomycin, 2 mM glutamine, and non-essential amino acids. EMT6 cells were cultured in Waymouth's medium supplemented with 15% FBS. All cells were maintained in a 37° C. humidified incubator with 5% CO₂.

### Cell Transfection

Plasmid HG10290-CH (Sino Biological Inc., Japan) harboring the C-His-tagged human Gal-1 cDNA and plasmid CV015, a pCMV3-C-His empty vector used in mock experiments (Sino Biological Inc.), were used for transient transfection. ES-2 cells were seeded at a density of 5×10³ cells/well in DMEM with 10% FBS in a 96-well plate, and the cells were incubated at 37° C. for 24 h. The cells were then transfected with the plasmid and respective controls using Lipofectamine 3000 for 72 h, according to the manufacturer's protocol.

### Western Blot

Cells were treated with GAT at concentrations ranging from 10 to 40 μg/mL or 10 μg/mL MG132 (M7449; Sigma-Aldrich, USA) for indicated times. They were then washed with phosphate-buffered saline (PBS) twice and resuspended in the radioimmunoprecipitation assay (RIPA) lysis buffer with protease inhibitor and 0.1 mM phenylmethylsulfonyl fluoride solution. Samples were collected and stored at −80° C. until use. Equal amounts of protein were subjected to sodium dodecyl sulfate-polyacrylamide gel electrophoresis and transferred to polyvinylidene fluoride membranes. The membranes were blocked with 1% bovine serum albumin in TBS-0.1% Tween-20 for 30 min, followed by incubation with a primary antibody, including anti-Gal-1 (GTX101566; GeneTex Inc., USA), anti-Gal-3 (ab76245; Abcam, USA), anti-pERK (9106S; Cell Signalling, USA), anti-ERK (4695; Cell Signalling), anti-glyceraldehyde-3-phosphate dehydrogenase (ab181602; Abcam), anti-ubiquitin (GT7811; GeneTex Inc.) and the appropriate horseradish peroxidase-conjugated secondary antibodies. The blots were visualized by chemiluminescence and quantified using an Amersham Imager 600 system (GE Healthcare, USA). The quantification of blotting results is based on the band intensity of each protein and is normalized to GAPDH.

### Immunoprecipitation

For immunoprecipitation, Protein A/G PLUS-Agarose Immunoprecipitation Reagent (Santa Cruz Biotechnology, CA, USA) was used to capture all proteins interacting with Gal-1. Briefly, Gal-1 antibodies (2 μg) were added to the cell lysates and incubated at 4° C. for 1 h. Subsequently, the Protein A/G PLUS-Agarose beads were introduced. The mixture was gently rotated at 4° C. for 24 h. The collected pellet was obtained through centrifugation at 3,000 rpm (approximately 1,000× g) for 5 minutes at 4° C. The pellet was then subjected to 2-4 washes with RIPA buffer. After washing, the supernatant was aspirated and discarded, and the pellet was resuspended in 40 μL of electrophoresis sample buffer. Subsequent steps involved electrophoresis and immunoblotting.

In vitro Proteasome Assay of Cell Lysates

The cells were rinsed with ice-cold PBS and scraped in proteasome lysis buffer (50 mM HEPES (pH 7.8), 10 mM NaCl, 1.5 mM MgCl₂, 1 mM EDTA, 250 mM sucrose, and 5 mM DTT) using cell scrapers. The cell lysates were sonicated twice for 10 seconds and centrifuged at 16,000× g for 10 mins at 4° C. The supernatants were transferred to fresh Eppendorf tubes and mixed with equal volumes of proteasome assay buffer (50 mM HEPES (pH 7.8), 10 mM NaCl, 1.5 mM MgCl₂, 1 mM EDTA, 250 mM sucrose, 5 mM DTT, and 10 mM ATP) (Maher, 2014). The mixture was then added with GAT and incubated at 37° C. for 72 h. Reactions were replenished with 2 mM ATP and collected every 24 h. The degraded samples were stored at −80 ° C. and analyzed by western blotting using the appropriate antibodies.

### Cell Cytotoxicity Assay

Cells were seeded at a density of 4×10³ cells/well in 96-well plates and cultured in accordance with the procedures described above. Triplicate wells of the cells were treated with GAT at 0, 6.25, 12.5, 25, and 50 μg/mL or 0.25% EtOH (solvent control) for 72 h. The cells were then subjected to 3-(4,5-dimethylthiazol-2-yl)-2,5-diphenyltetrazolium bromide (MTT) assay (TACS; Trevigen, USA) and incubated at 37° C. for 4 h. The absorbance at 570 nm was measured using a microplate reader (CLARIOstarPlus; BMG LABTECH, Germany). The IC₅₀ values were calculated using linear regression of the percentage survival rate versus drug concentration. The viability of transfected cells was detected at a GAT concentration of 50μg/mL.

The drug combination effects were calculated using the combination index (CI) (Chou, 2010) method with the CompuSyn software (Zhang et al., 2016). CI<1, =1, and >1 indicate synergistic, additive, and antagonistic effects, respectively.

### Immunohistochemistry (IHC)

The tissues were fixed in 10% formaldehyde and embedded in paraffin. The immunohistochemical detection of Gal-1 (GTX101566; GeneTex Inc.), rabbit polyclonal α-SMA (ab5694; Abcam) and CD8 (ab203035; Abcam) antibodies were using a fully automated IHC stainer (Bond-III; Leica Biosystems, Wetzlar, Germany). Briefly, antigen retrieval was carried out at 100° C. for 20 min. After blocking the endogenous peroxidase activity for 5 min, a primary antibody, including Gal-1, α-SMA, and CD8 antibodies, was incubated for 50 min at room temperature at a dilution of 1:500, 1:300, and 1:250. The samples were visualized using diaminobenzidine and counterstained with hematoxylin. Whole slide scanning was done by PANNORAMIC MIDI II (3DHISTECH Ltd., Hungary), and the Images were exported by PANNORAMIC Viewer (3DHISTECH Ltd). The positive area of α-SMA and the intensity of Gal-1 were detected and estimated using ZEN Microscopy Software (Carl Zeiss Corporation, Germany).

### Hematoxylin and Eosin (H&E) Staining

Histopathology of the tumor tissues was evaluated in paraffin-embedded sections using a Leica ST5010 Autostainer XL stainer. Briefly, the paraffin sections were deparaffinized in xylene and rehydrated using an ethanol gradient. The sections were washed with tap water and pretreated with hematoxylin (Gill's II solution; Leica) for 4 min, followed by eosin (Gill's II solution; Leica) for 4 min. Slide sections were mounted and covered with coverslips. Whole slide scanning was done by PANNORAMIC MIDI II (3DHISTECH LTD.).

In vivo Xenograft Tumor Assessment

All the mice were purchased from LASCO Laboratory (BioLASCO Taiwan, Taiwan). For the orthotopic ovarian cancer model, six-to eight-week-old female NPG (NOD-Prkdc^{scid}IL2^{null}) mice were used. NPG is a strain of immunodeficient mice based on the NOD/SCID background, with an additional deletion of the IL2rg gene that leads to the incompetence of T, B, and NK cell functions, and is more suitable for establishing immune humanized mice than NOD/SCID. For the EMT6 syngeneic mammary tumor model, six-to eight-week-old female BALB/cAnNCrlBltw mice were used. Mice were kept at specific pathogen-free conditions under a 12:12 light-dark cycle with food and water provided ad libitum; ambient temperature and relative humidity were set at 22±3°3 and 50±20%, respectively. At the endpoint, mice were sacrificed by CO₂ euthanasia.

In the orthotopic ovarian cancer model, animals were anesthetized using isoflurane. A left-side incision approximately 1.0 cm in length was made lateral to the midline of the lower back, exposing the ovary beneath the muscle layer. The left ovary was carefully extracted to reveal the bursa. In vitro cultured ES-2 cells were suspended at a concentration of 1×10⁵cells/10 μL and injected into the left ovarian bursa using a 29 G syringe needle, ensuring no leakage occurred. Subsequently, the ovary was repositioned; if no bleeding was observed, the abdomen and skin were closed using surgical sutures. One week before tumor injection, a humanized peripheral blood mononuclear cell (Hu-PBMC)-mice model was established via intravenous injection of 1×10⁷ human peripheral blood mononuclear cells (PBMCs) into NPG mice or NOD-scid-IL2Rγ^{null} mice. GAT (100 mg/kg) was administered daily via oral gavage. Paclitaxel (20 mg/kg) was administered once weekly via intravenous injection. In the experiment using humanized PBMC mice, each group consisted of three mice. In the experiment investigating the combination effect of GAT and Paclitaxel, each group consisted of four mice. In the EMT6 syngeneic mammary tumor model, 5×10⁵ EMT6 cells in 0.1 mL were injected into the right flank of female BALB/c mice. The animals received treatment either intravenously or intraperitoneally with 20 mg/kg of GAT twice a week or 10 mg/kg of anti-PD-L1 antibody (Atezolizumab, purchased from Biointron Biological Inc.) once a week. Each group consisted of four mice. The animals in the control and anti-PD-L1 antibody groups were injected with the same solvent used for GAT as the vehicle control. Measurements were performed thrice a week from the day of inoculation. Tumor volume was calculated using the ellipsoid equation according to the records ((length×width×width)/2). At the end of the study, the mice were sacrificed, and the tumors were excised and weighed. The orthotopic ovarian tumor samples were subjected to tissue sectioning, H&E staining, IHC (α-SMA, Gal-1), and TILs analysis. The EMT6 mammary tumor samples underwent tissue sectioning, H&E staining, and IHC (α-SMA, Gal-1, CD8).

### Flow Cytometry Analysis and Quantification of TILs

To determine the percentage of tumor tissues containing a lymphocytic infiltrate, the infiltration of CD45⁺ immune cells in a humanized mouse model was analyzed using flow cytometry (Shultz et al., 2012). The average percentage of TILs in the tumors was measured in Hu-PBMC with or without GAT administration. Briefly, tumor tissues were first treated with 10 U/mL collagenase type IV at 37° C. for 2 h. Single-cell suspensions prepared by filtering through a 70-μm nylon strainer were incubated for 30 min on ice with Fc Receptor Binding Inhibitor antibody. The cells were then incubated for 15 min in PBS containing 1.0 mM EDTA with a 1:500 dilution of PE/Cy 5 anti-human CD 45 antibody at room temperature for 30 min. The cells were washed twice with PBS and analyzed using a CytoFLEX Flow Cytometer (CytoFLEX; Beckman). The results were analyzed using CytExpert 2.3 software (Beckman).

### Paclitaxel Content Analysis

Tumor tissues were homogenized in PBS at a ratio of 5:1 (PBS: tissue weight). The homogenates were mixed with acetonitrile at a ratio of 3:1 (acetonitrile: homogenate) to precipitate proteins. After sonication and centrifugation at approximately 15,000 rpm and 4° for 5 min, the supernatants were collected and injected into the AB SCIEX QTRAP 5500 LC-MS/MS system. Chromatographic separation was achieved using a Zorbax RRHD Eclipse plus C18 column (3×100 mm, 1.9μm) with Exion AD30 Ultra Performance Liquid Chromatography. Paclitaxel was quantified based on the peak area of the MS/MS transition 854.4 to 286.1.

### Statistical Analysis

Data are represented as the mean±standard deviation. Statistical analysis were conducted via one-way analysis of variance with Turkey's honest significant difference post-hoc for multiple comparisons or Student's t-test for two-sample comparisons to assess the statistical significance. Statistical significance was set at P<0.05.

### Example 1: Preparation and Characterization of GAT from G. lucidum

### 1.1 Preparation

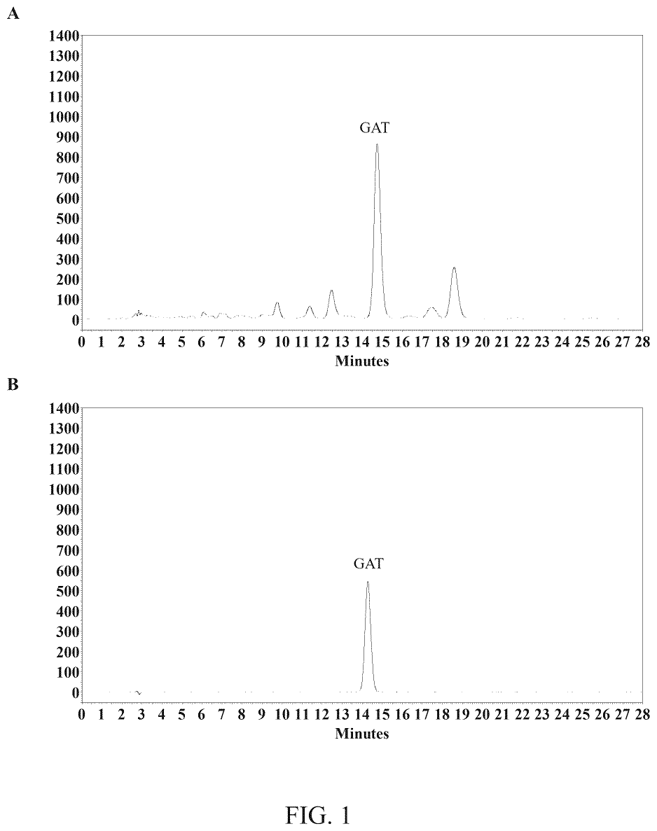

GAT was prepared in accordance with procedures described in the “Materials and methods” section and was characterized by HPLC. Results are provided in FIG. 1.

HPLC revealed multiple eluted peaks in the crude ganoderic acid extract (FIG. 1A), whereas the purified form only exhibited a single peak (FIG. 1B), which was identified as GAT.

### 1.2 Characterization

### 1.2.1 Cytotoxicity

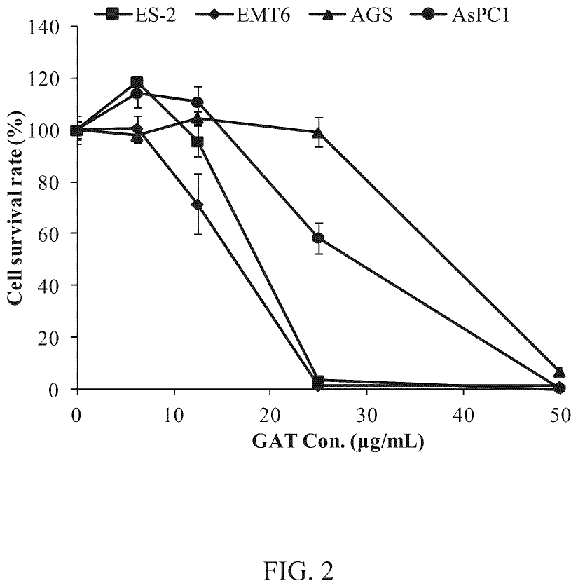

Human ovarian (ES-2), pancreatic (AsPC1), gastric (AGS), and mouse mammary (EMT6) cancer cell lines were used to assess GAT cytotoxicity in cancer cells. MTT assay was used to determine the survival rates of these cancer cells after treatment with increasing concentrations of GAT for 72 h (FIG. 2). GAT exhibited cytotoxicity against all cancer cells, exhibiting half maximal inhibitory concentration (IC 50) values of 17.6, 13.7, 28.7, and 38.3 μg/mL in ES-2, EMT6, AsPC1, and AGS cells, respectively. Further, no solvent-related toxicity was observed in these cell lines at 0.25% ethanol (equivalent to the ethanol concentration at a GAT dosage of 50 μg/mL) (data not shown). Specifically, ES-2 human ovarian cancer and EMT6 mouse mammary carcinoma cell lines exhibited higher sensitivity to GAT than the other cell lines and were selected for subsequent experiments

### 1.22 GAT decreases α-SMA⁺ Cell Proportion and Increases Immune Cell Infiltration in the Tumor Microenvironment (TME)

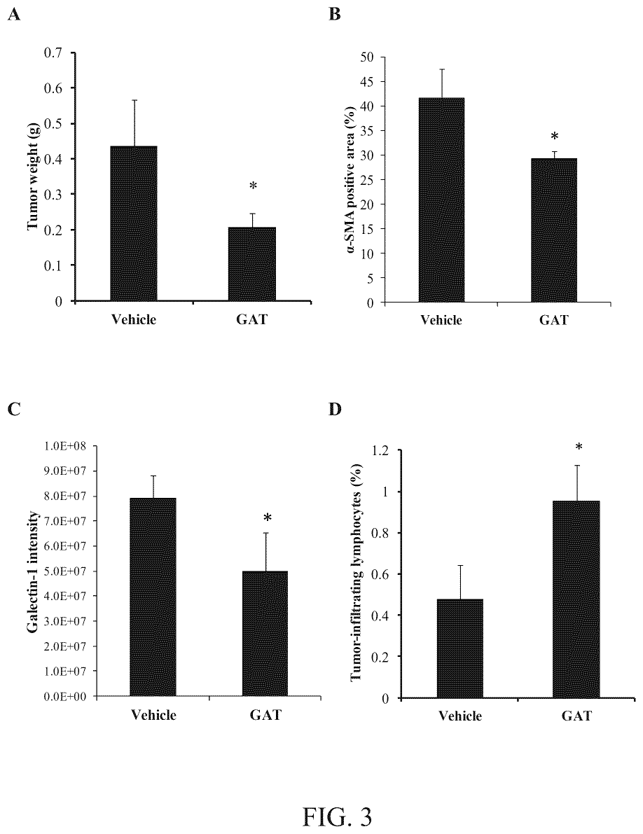

To evaluate anti-tumor effects in vivo, an orthotopic ovarian cancer model in PBMC humanized mice was established in accordance with procedures described in “Materials and methods” section. Tumor weight analysis revealed that GAT significantly inhibited tumor growth, with an inhibition rate of 52% (FIG. 3A). Immunohistochemical staining revealed that GAT reduced the α-SMA+area (FIG. 3B), which serves as a biomarker for CAFs, indicating a decrease in the stromal proportion within the tumor, as well as the level of galectin-1 (Gal-1) (FIG. 3C).

Human CD45, also known as leukocyte common antigen, was used to analyze engrafted cells in PBMC humanized mice, where the majority (>98%) were T cells. Treatment with GAT notably increased the proportion of human CD45⁺ cells within the tumor, from an average of 0.48% to 0.96% (FIG. 3D). The increase in TILs was consistent with the observed inhibition of tumor growth. These findings suggest that the tumor-suppressive effect of GAT involves the significant reduction of the α-SMA⁺ cell proportion and enhanced T-cell infiltration within the tumor, indicative of TME remodeling.

### 1.2.3 GAT Downregulates Gal-1 Expression and Suppresses the Extracellular Signal-Regulated Kinase Pathway

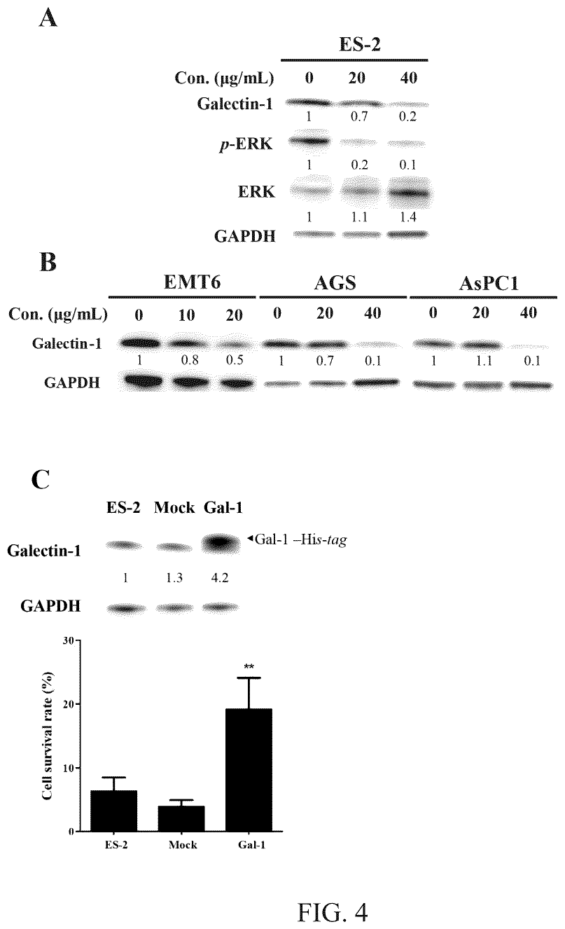

Suppression of Gal-1 protein expression by GAT was validated via western blotting analysis A dose-dependent downregulation of Gal-1 levels in ES-2 cells EMT6, AGS, and AsPC1 cells were observed, respectively (FIGS. 4, A and B). Additionally, the downstream regulatory target of Gal-1, the extracellular signal-regulated kinase (ERK) signaling pathway, which regulates cell survival and migration, was examined after GAT treatment. It was found that respective levels of Gal-1 and phosphorylated-ERK decreased after being treated with 20 and 40 μg/mL GAT (FIG. 4A). Furthermore, downregulation of Gal-1 by GAT was confirmed by IHC in orthotopic ovarian tumor tissues (FIG. 3C). To assess the specificity of Gal-1 as a target of GAT-mediated cellular cytotoxicity, transient overexpression of Gal-1 was performed in ES-2 cells, which led to a four-fold increase in Gal-1 expression in ES-2 cells (ES-2 -Gal-1) compared to that in mock cells transfected with a blank vector and non-transfected ES-2 cells (FIG. 3). Notably, the survival rate of ES-2-Gal-1 cells in the presence of GAT at 50 μg/mL significantly increased by 15.3% compared to the mock ES-2 cells (FIG. 4C). These results suggest Gal-1 as a potential target protein involved in the molecular mechanisms of GAT.

### 1.2.4 GAT Inhibits Gal-1 Expression via Ubiquitination-Induced Protein Degradation

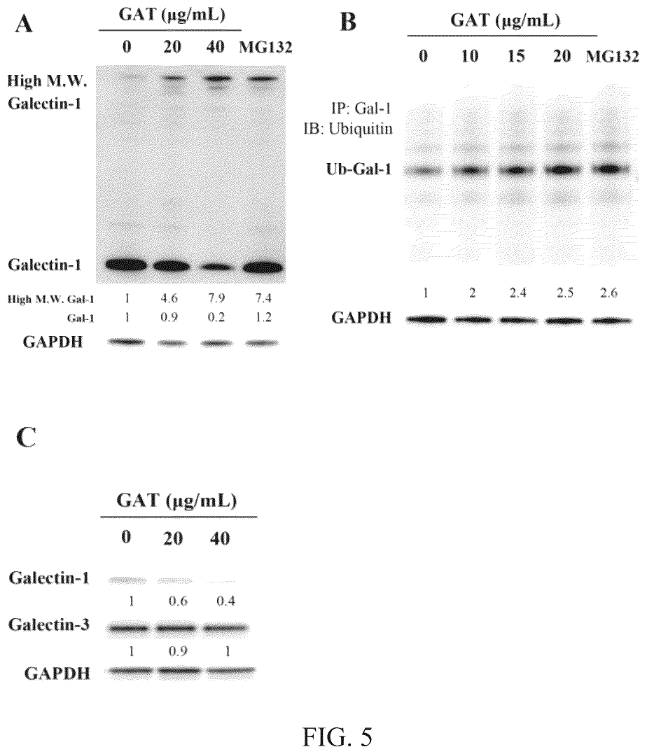

In western blotting analysis, treatment with GAT decreased Gal-1 (15 kd) and increased the band with a high molecular weight on the same blotting (FIG. 5A). MG132 is a proteasome inhibitor that induces accumulation of polyubiquitination products in cell. Upon treatment with MG132, the intensity of the high molecular weight band increased, indicating that the product was poly poly-ubiquitination-Gal-1 (FIG. 5A). To explore whether GAT facilitates the ubiquitination of Gal-1, we conducted an immunoprecipitation experiment to capture all proteins associated with Gal-1 and performed a ubiquitination assay. These results indicated a notable dose-dependent enhancement in the ubiquitination of Gal-1 protein induced by GAT (FIG. 5B). Treatment with MG132 also led to an increase in Ubiquitin-Gal-1, consistent with the observations shown in FIG. 5A. To evaluate the GAT-mediated protein degradation, the in vitro degradation assay was performed. In the GAT incubated with the ES-2 cell extracts and reaction buffers, Gal-1 levels decreased in a dose-dependent manner, but not Gal-3, a galectin family member that is shown to be associated with tumor growth and progression. The results demonstrated that GAT was capable of inducing proteasomal degradation of Gal-1 and displayed no noteworthy alterations in Gal-3 levels upon GAT treatment, signifying the selective suppression of Gal-1 by GAT (FIG. 5C). These data indicate that GAT reduces Gal-1 expression via ubiquitination-induced degradation

### Example 2: GAT Enhanced Anti-Tumor Effects of Paclitaxel

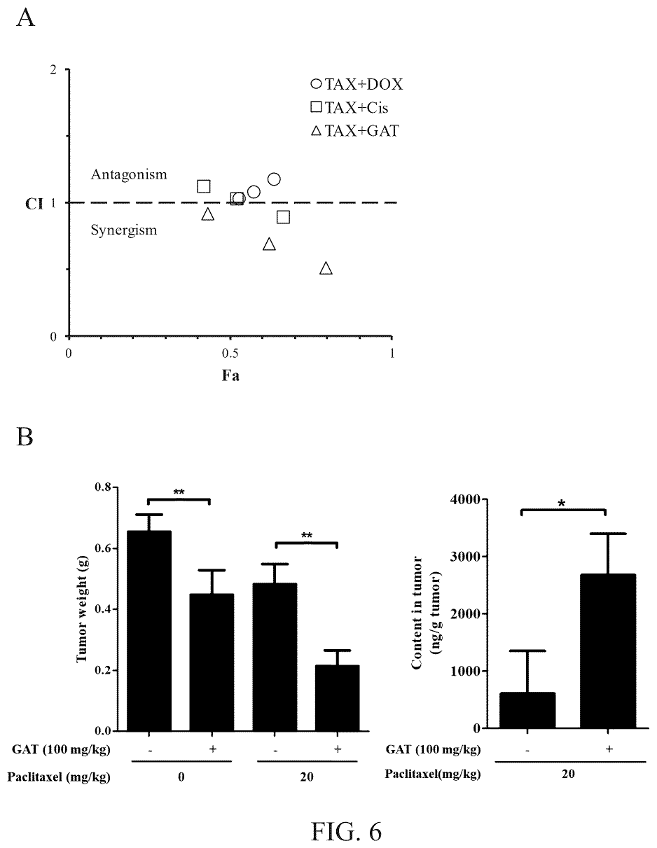

In this example, the effects of GAT in combination with paclitaxel on ovarian cancer ES-2 cells were evulated by using MTT assay and the combination index (CI) method, in which CI values<1, =1, and >1 indicated synergistic, additive, and antagonistic effects, respectively. Results are provided in FIG. 6.

As shown in FIG. 6A, the combination of GAT and paclitaxel exhibited a synergistic effect with a CI value of <1. Conversely, the CI value for the combination of cisplatin and paclitaxel was close to 1 (additive) and the CI value of the combination of paclitaxel and doxorubicin was higher than 1 (antagonism). To validate the in vitro observation that GAT and paclitaxel exerted anti-tumor effects synergistically, in vivo tests were performed by using an orthotopic ovarian tumor model. Treatment with GAT significantly reduced tumor weight compared to that in the untreated group. The average tumor weights in the GAT, paclitaxel, and combination groups were 0.43 g, 0.48 g, and 0.21 g, respectively (FIG. 6B). GAT or paclitaxel alone exhibited similar inhibition of in vivo tumor responses. However, the combination of paclitaxel and GAT significantly suppressed tumor growth compared with paclitaxel alone (FIG. 6B). Furthermore, quantification of paclitaxel revealed that GAT significantly enhanced the paclitaxel content in tumor tissues (FIG. 6B). These findings indicated that GAT potentiates the anti-tumor effects of paclitaxel, leading to a more efficacious treatment of ovarian cancer.

### Example 3: GAT Significantly Improves the Response to Anti-PD-L1 Immunotherapy

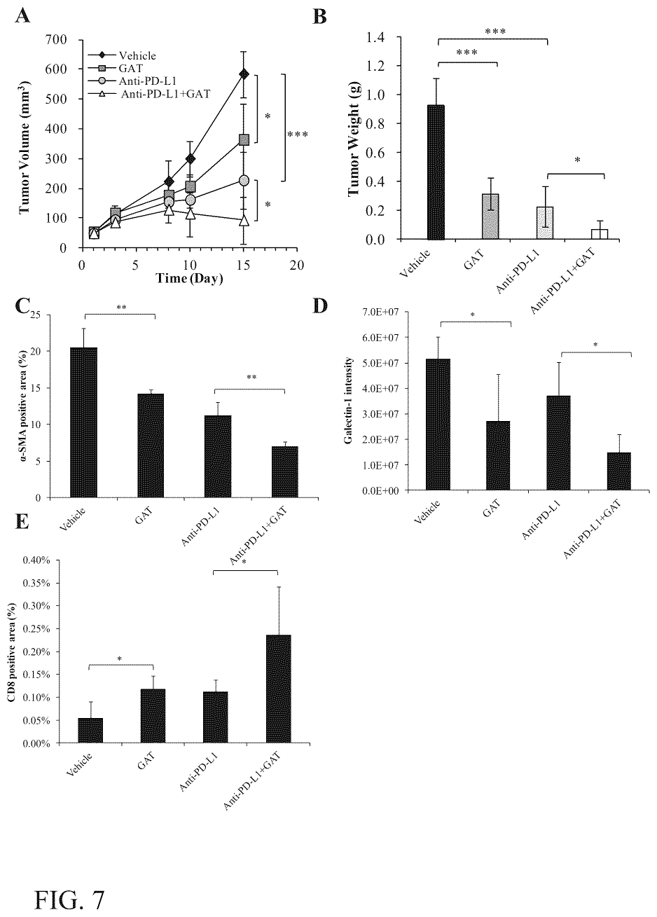

In this example, the efficacy of the combination of GAT and an anti-PD-L1 antibody (atezolizumab) was evulated by using the EMT6 syngeneic mammary cancer model. The syngeneic model is suitable for studying the interactions among immune cells, cancer cells, and the TME in immunotherapy while avoiding graft versus host disease. Results are provided in FIG. 7.

It was found that treatment with GAT or anti-PD-L1 antibody alone significantly inhibited tumor growth (FIG. 7A). However, the combination of GAT and the anti-PD-L1 antibody exhibited the most potent anti-tumor response, resulting in the smallest tumor volume and weight compared to the other treatment groups (FIG. 7B). Additionally, treatment with GAT was found to reduce the proportion of α-SMA⁺ stromal cells and Gal-1 content in tumor tissues (FIGS. 7D and 7D), which is consistent with our findings in the ovarian cancer model (FIG. 3).

Since PD-L1 and Gal-1 influence the composition of TILs in TME, particularly CD8⁺ cells, through different regulatory mechanisms. Therefore, intratumoral T cell infiltration, specifically CD8⁺ T cells infiltration was investigated. The results indicated that GAT treatment increased intratumoral T cell infiltration (FIG. 7E), leading to an improved response to anti-PD-L1 therapy. These results suggested that the combination treatment of the PD-L1 inhibitor and GAT could be a more effective therapeutic strategy, enhancing anti-tumor responses in the EMT6 mammary cancer model.

Taken together, results of the present disclosure indicate that GAT effectively inhibited tunor growth both in vitro and in vivo. Further, GAT enhanced the efficacy of paclitaxel treatment and anti-PD-L1 immunotherapy. These results confirmed GAT's potential as a candidate for the development of a therapeutic agent for treating cancer and/ot ehnancing the efficacy of existing cancer therapies.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

What is claimed is:

1. A method of treating a cancer in a subject comprising administering to the subject an effective amount of a combination of ganoderic acid T (GAT) and an anti-cancer agent, wherein the administration of the combination of GAT and the anti-cancer agent results in a synergistic suppression of the growth of the cancer as compared to that achieved by the anti-cancer agent alone.

2. The method of claim 1, wherein the GAT is administered before, together with, or after administering the anti-cancer agent.

3. The method of claim 1, wherein the cancer is glioblastoma, thyroid cancer, gastrointestinal cancer, liver cancer, lung cancer, breast cancer, pancreatic cancer, melanoma, prostate cancer, ovarian cancer, adenocarcinoma, bladder cancer or kidney cancer.

4. The method of claim 3, wherein the cancer is ovarian cancer.

5. The method of claim 3, wherein the cancer is breast cancer.

6. The method of claim 1, wherein the anti-cancer agent is a chemotherapeutic agent or anti-PD-L1 antibody.

7. The method of claim 6, wherein the chemotherapeutic agent is selected from the group consisting of paclitaxel, docetaxel, camptothecin (CPT), topotecan (TPT), irinotecan (CPT-11), doxorubicin, daunorubicin, epirubicin, fluorouracil, cisplatin, cyclophosphamide, vinblastine, vincristine, ifosfamide, melphalan, mitomycin, methotrexate, mitoxantrone, teniposide, etoposide, bleomycin, leucovorin, cytarabine, dactinomycin, streptozocin, and combretastatin A4-phosphate.

8. The method of claim 7, wherein the chemotherapeutic agent is paclitaxel.

9. The method of claim 6, wherein the anti-PD-L1 antibody is atezolizumab.

10. The method of claim 8, wherein the GAT and paclitaxel are respectively administered 8 mg/Kg and 2 mg/Kg to the subject.

11. The method of claim 9, wherein the GAT and atezolizumab are respectively administered at 8 mg/Kg and 0.8 mg/Kg to the subject.

12. The method of claim 1, wherein the subject is a human.
